(19) United States
(12) Patent Application Publication
(10) Pub. No.: US 2014/0127133 A1
(43) Pub. Date: May 8, 2014

(54) METHOD FOR EVALUATING A MENTAL DISORDER

(71) Applicants: Claude Brodski, Tel Aviv (IL); Ora Kofman, Beer Sheva (IL); Marin Jukic, Belgrade (RS)

(72) Inventors: Claude Brodski, Tel Aviv (IL); Ora Kofman, Beer Sheva (IL); Marin Jukic, Belgrade (RS)

(21) Appl. No.: 13/977,951

(22) PCT Filed: Nov. 13, 2011
(86) PCT No.: PCT/IL11/00874

§ 371 Date: Sep. 29, 2013

Related U.S. Application Data
(60) Provisional application No. 61/435,373, filed on Jan. 24, 2011.

(51) Int. Cl. A61K 49/00

(52) U.S. Cl. CPC A61K 49/00
USPC 424/9.2

(57) ABSTRACT

A method for evaluating the efficacy of a candidate drug for the treatment of a bipolar disorder is provided. The method includes administering the candidate drug or combination of drugs to a mouse characterized by an elevated amount of Otx2 protein in the hindbrain; and evaluating the extent, frequency and/or reversal of a mouse behaviour that is homologous to a bipolar disorder in a human.

A method for evaluating the efficacy of a substance or a combination of substances in mice having an elevated amount of Otx2 protein in the hindbrain for the treatment of a bipolar disorder in a subject is provided.

### BACKGROUND OF THE INVENTION

Bipolar disorder (BD), also known as manic depressive disorder, is a severe psychiatric condition that affects approximately on to two percent of the general population. However, the treatment for this disease is often insufficient. This is, at least in part, due to the lack of suitable animal models. Genetic predisposition, dysfunction of monoaminergic neurons and altered signaling pathways are each thought to play a critical role in bipolar disorder. However, how these factors interact in the pathophysiology and pharmacotherapy of this disease is poorly understood

Although no single gene mutation has been shown to be causative, heritability is known to play a significant role in the etiology of BD. Genetic studies have consistently suggested genes involved in monoaminergic neurotransmission (e.g., SLC6A4, TPH2, DRD4, SLC6A3), signaling pathways (DISC1) and neurodevelopment (BDNF and NRG1) as susceptibility genes for bipolar disorder.

The molecular mechanisms underlying the therapeutic effects of mood stabilizers such as lithium and valproate are still not well understood. However, several lines of evidence suggest that lithium's and valproate's inhibition of glycogen synthase kinase (GSK)-3 and of inositol signaling mediates the drugs' effectiveness. These inhibitory characteristics might imply that in bipolar patients, one or both of these pathways are overactive. The shared characteristic of most neuroleptics to block dopamine receptors and the common effect of antidepressants to increase serotonergic and noradrenergic neurotransmission in particular brain regions support the hypothesis that an imbalance of monoaminergic neurotransmission is involved in the pathophysiology of BD.

Bipolar disorder is classified according to the pattern and severity of the symptoms as bipolar disorder I, bipolar disorder II, or cyclothymic disorder. Patients with one type may develop another. Nevertheless, they are sufficiently distinct to merit separate classifications. Bipolar disorder I is characterized by at least one manic episode or mixed episode (symptoms of both mania and depression occurring simultaneously), and one or more depressive episodes, that lasts for at least a week. In most cases, manic episodes precede or follow depressive episodes in a regular pattern. Episodes are more acute and severe than in the other two categories.

Without treatment, patients average four episodes of dysregulated mood each year. With mania, either euphoria or irritability may mark the phase. In addition, there are significant negative effects (such as sexual recklessness, excessive and impulsive shopping, and sudden traveling) on a patient's social life, performance at work, or both. Untreated mania lasts at least a week, and it can last for months. Typically, depressive episodes tend to last 6-12 months, if left untreated.

Bipolar disorder II is characterized by episodes of predominantly major depressive symptoms, with occasional episodes of hypomania, which last for at least 4 days. Hypomania is similar to mania, but the symptoms (typically euphoria) are less severe and do not last as long.

Patients do not experience manic or mixed episodes, and most return to fully functional levels between episodes. However, bipolar II patients have a more chronic course, significantly more depressive episodes, and shorter periods of being well between episodes than patients with type I have. It is highly associated with the risk for suicide.

The lack of an animal model for bipolar disorder is a significant rate limiting step in the investigation of the neurobiology of BD. Although different animal models have been suggested, there is presently no animal model available that mimics inter- as well as intra-individual behavioural variability which is a hallmark of BD.

### SUMMARY OF THE INVENTION

In one embodiment, the present invention provides a method for evaluating the efficacy of a substance or a combination of substances for the treatment of a bipolar disorder in a subject, comprising the steps of a. administering the substance or the combination of substances to a mouse comprising an elevated amount of Otx2 protein in the hindbrain; and b. evaluating manic and depressive-like behaviour in the mouse.

### BRIEF DESCRIPTION OF THE DRAWINGS

FIG. 1. A bar graph showing that acute lithium treatment attenuates locomotor activity of En1^{+/Otx2} mice (hereinafter “Otx2”). Wild-Type (WT) (n=10) and En1^{+/Otx2} mutant mice (n=10) were exposed to an acute lithium treatment (150 mg/kg). Locomotor activity was measured immediately after injection for a time period of 50 minutes. Mutants showed a significant reduction in locomotor activity compared to WT (t=−3.14987; p<0.0062).

FIG. 2. Bar graphs showing that En1^{+/Otx2} mutants have an increase in GSK3 in the hippocampus, a decrease in pERK2 protein levels in the hippocampus and a decrease in pERK1 and pERK2 protein levels prefrontal cortex. According to FIG. 2A, GSK-3beta protein levels were increased in mutants compared to WT as measured by Western blotting using a one-tailed t-test the increase is statistically significant. ((p=0.04), n=5 in each group). According to FIG. 2B, pERK2 levels are 26% decreased in En1^{+/Otx2} mutants compared to WT as measured by Western blotting ((p<0.05). n=3 in each group). According to FIG. 2C, pERK1 and pERK2 levels are decreased in En1^{+/Otx2} mutants compared to WT as measured by Western blotting.

FIG. 3. A bar graph showing that chronic lithium treatment leads to a reduction of hyperactivity in En1^{+/Otx2} mutants. Locomotor activity of mice was assessed in the open field (pre Li). High activity mice and low activity mice were treated for 5 days with 0.2 mg/kg lithium in their ground food. Subsequently they were treated for 10 days with 0.4 mg/kg lithium. At the end of the treatment period, locomotor activity was assessed for 30 minutes in the open field. Lithium exposure in En1^{+/Otx2} mice lead to a significant reduction in locomotor activity in hyperactive En1^{+/Otx2} (p<0.05), in contrast to the highly active WT mice. The low active mice were not significantly affected by lithium treatment.

FIG. 4. Plots showing that hyperactive En1^{+/Otx2} mutants spent more time in the center of the open field, suggesting reduced levels of anxiety/risk taking behaviour. When treated with saline, there was a significant correlation between the ratio of time spent in the center/in the periphery and the total level of activity (r=0.68, <0.01) in the En1^{+/Otx2} mutant mice (4A) but not the WT mice (4B) (r=0.002, P=0.99) suggesting that the hyperactivity of the mutants is accompanied by reduced anxiety.

FIG. 5. The time Otx2 mutants spend in the center of the open field was correlated with their locomotor activity levels. Time Otx2 mutants spend in the center of the open field correlates with their locomotor activity level. (p<0.001), in contrast to WT.

FIG. 6. Three graphs showing that En1^{+/Otx2} mutants have a higher intra-individual variability in locomotor activity overtime. Animals were placed during a period of 12 days 4 times in the open field and locomotor activity was measured for 30 minutes (6A for WT and 6B for mutants). En1^{+/Otx2} show a higher intra-individual variability compared to WT as shown by a significant increase in the coefficient of variance of mutants compared to WT (6C).

FIG. 7. Graphs showing that En1^{+/Otx2} mutants show a higher intra-individual variability in sugar consumption over time 7A shows preferences of WT and En1^{+/Otx2} mice for 8% sucrose versus tap water as a measure of fluid consumption. After 12 hours of water deprivation, 10 WT (7B) and 10 En1^{+/Otx2} (7C) male mice were given access to tap water in one bottle or to an 8% sucrose solution in another bottle, and fluid consumption measured. This procedure was repeated for 6 days. En1^{+/Otx2} mice showed a higher intra-individual variability compared to WT as shown by a significant increase in the coefficient of variance of mutants compared to WT (7D). Asterisks indicate significant difference in t-testing (P<0.05).

FIG. 8. Alterations in monoaminergic neurons in adult Otx2 mutants. Dopamine transporter (Dat), Serotonin transporter (Sert), and tyrosine hydroxylase (TH) were measured using immunohistochemistry to identify cells expressing dopamine, serotonin, and norepinephrine, respectively in Otx2 mutant mice compared to wild type mice.

FIG. 9. Otx2 mutants and wild type mice were evaluated for manic-like behaviour in the open field test for 60 min with or without a 2 mg/kg or 4 mg/kg administration of amphetamine.

FIG. 10. Otx2 and wild type (WT) animals were evaluated for hyperactivity in the open field test. 30 Otx2 and 30 wild type (WT) animals were recorded for 45 min in the open field during both light and dark phase. Otx2 mutants were hyperactive (F=40.889, p<0.001) in both phases and showed more inter-individual variation (A). Distances traveled in the light phase correlated with the distances traveled in the dark phase for both WT (B) and Otx2 animals (C).

FIG. 11. Measures of activity (left graph) in movements per minute and body temperature (right graph) of Otx2 mutant mice compared to wild-type mice over the course of the light-dark cycle. Six WT and 7 Otx2 animals were chronically implanted with activity and body temperature monitor probes. Otx2 animals show increased home cage activity in the dark phase (p<0.001 for all) (A). In the light phase, Otx2 mutants are only more active in the first quartile (t=−2.776, p=0.008) suggesting that mutants sleep less in this period. Hyperactivity of mutants is not correlated with increased temperature. In the light phase mutants showed a decrease in temperature (p<0.001 for the last three quartiles) (B).

FIG. 12. Olanzapine reduced hyperactivity in Otx2 mutants as well as the activity of WT. 60 min. after receiving a subcutaneous injection containing either saline or 1 mg/kg olanzapine, locomotor activity of animals (n=10 in each group) was assessed in the open field for 60 min. Olanzapine decreased activity of both WT and 01×2 animals (F=4.834, p=0.034).

FIG. 13. Otx2 mutants show more entries into the open arm in the elevated plus maze, which can be reversed by olanzapine. 10 WT and 10 Otx2 animals were placed in the center of the elevated plus maze facing the closed arm, and their behaviour was recorded for 5 min, Otx2 animals showed higher percentage of entries into open arms compared to WT (t=−3.174, p=0.005). Sixty minutes after administration of olanzapine or saline, animals were placed in the center of the elevated plus maze facing the closed arm, and their behaviour was recorded for 5 min. As measurement for risk-taking behaviour in the Elevated Plus Maze (EPM), percentage of entries into and percentage of time spent in open arms were measured. The number of entries to all arms of the EPM was taken as the measurement of general activity of animals. Otx2 animals showed a higher percentage of entries into open arms then the WT (A), which olanzapine specifically reduced (F_{Genotype×Treatment}=4.663, p=0.038). Reduction of time spent in open arms of the EPM didn't reach statistical significance (13). Olanzapine decreased general activity of both groups (F=6.065, p=0.018), with no difference between groups (C).

FIG. 14. Otx2 mutants showed more risk taking behaviour in Dark-Light box, which can be reversed by olanzapine. 10 WT and 10 Otx2 animals were introduced to the center of the light part of the dark-light box and were recorded for 10 minutes. Otx2 animals showed increased time spent in the light part of the apparatus compared to WT (t=−2.693, p=0.015). We conclude that Otx2 mice show increased risk-taking behaviour. Sixty minutes after treatment, animals were introduced to the center of the light part of the dark-light box and were recorded for 10 minutes. More time spent in illuminated part indicated more risk taking behaviour. Saline treated Otx2 animals showed increased time spent in the light part of the apparatus compared to WT (t=−2.693, p=0.015). Olanzapine reduced the time Otx2 mutants spent in the light compartment, and increased the time WT animals were present in the light compartment (F_{Genotype×Treatment}=2.886, p=0.098).

FIG. 15. Olanzapine increases immobile time in the forced swim test (FST) for both Otx2 and WT. Sixty minutes after treatment, animals were introduced to FST and recorded for 6 min. Videos were analyzed only for the last 4 min of trial. Otx2 mutants showed a trend to spend less time immobile (F=2.901, p=0.098) while olanzapine increased immobile time for both WT and Otx2 animals (F=18.03, p<0.001).

### DETAILED DESCRIPTION OF THE INVENTION

In one embodiment, the present invention provides an animal model for testing the efficiency of a substance in reversing or alleviating a manic and/or depressive-like behaviour in mice (animal model). In one embodiment, the present invention provides a method for evaluating the efficacy of a substance or a combination of substances for the treatment of a bipolar disorder in a subject, comprising the steps of a. administering the substance or a combination of substances to a mouse mutant ectopically expressing Otx2 in the hindbrain; and b. evaluating manic and depressive-like behaviour in the mouse. In one embodiment, the present invention provides a method for evaluating the efficacy of a substance or a combination of substances in mice ectopically expressing Otx2 in the hindbrain for the treatment of a bipolar disorder in a subject, comprising the steps of a. administering the substance or a combination of substances to a mouse mutant ectopically expressing Otx2 in the hindbrain; and b. evaluating manic and depressive-like behaviour in the mouse.

In another embodiment, the present invention provides methods which include administering a candidate drug or a candidate combination of drugs to a mouse characterized by an elevated amount of Otx2 protein in the hindbrain; and evaluating the extent, frequency and/or reversal of a mouse behaviour that is homologous to a bipolar disorder in a human. In another embodiment, the present invention provides methods based on a mouse having an elevated amount of Otx2 protein in the hindbrain, which include evaluating/assessing the efficacy of a candidate drug or a candidate combination of drugs in ameliorating, alleviating, reducing the frequency, inhibiting or reversing a mouse behaviour that is homologous to a bipolar disorder in a human.

### Evaluating Efficacy

In another embodiment, the present invention provides a method for evaluating the in vivo toxicity and/or side effects of a substance or a combination of substances for use in treatment of a bipolar disorder in a subject, comprising the steps of: a. administering the substance or a combination of substances to a mouse mutant ectopically expressing Otx2 in the hindbrain; b. evaluating manic and depressive-like behaviour like behaviour in the mouse; and c. evaluating the toxicity in the mouse. In another embodiment, methods for evaluating toxicity of therapeutic substances are known to one of skill in the art. In another embodiment, methods for evaluating toxicity of therapeutic substances according to the invention further include a decline in a bipolar disorder behaviour.

In another embodiment, the term “evaluating” according to the invention includes assessing. In another embodiment, the term “evaluating” according to the invention includes estimating. In another embodiment, the phrase “evaluating the efficacy of a substance or a combination of substances” includes screening a substance or a favorable combination of substances. In another embodiment, the phrase “evaluating the efficacy” is measuring known behaviours associated with bipolar disorders or schizophrenia. In another embodiment, the process for “evaluating the efficacy” as described herein results in alleviation of at least one symptom associated with a bipolar disorder or schizophrenia. In another embodiment, a substance or a combination of substances are said to be efficacious when at least one behaviour or symptom associated with a bipolar disorder is alleviated or diminished. In another embodiment, a substance or a combination of substances are said to be efficacious when at least one behaviour or symptom associated with a bipolar disorder is reversed. In another embodiment, provided herein a method for identifying substances that reverse aberrant behaviour associated with a bipolar disorder or schizophrenia. In another embodiment, provided herein a method for identifying substances that alleviate at least one symptom associated with a bipolar disorder or schizophrenia. In another embodiment, provided herein a method for identifying the safety measures that should be taken by a subject treated with a compound having both a desired therapeutic impact but also unwanted side effects. In another embodiment, provided herein a method for identifying and treating unwanted side effects of a psychiatric drug.

In another embodiment, a substance or a combination of substances are said to be inefficacious, for example, in instances wherein at least one behaviour or symptom associated with a bipolar disorder is worsened. In another embodiment, a substance or a combination of substances are said to be inefficacious, for example, in instances wherein the substance or a combination of substances have at least one devastating effect that outweigh any benefit associated with the substance or the combination of substances. In another embodiment, a devastating effect includes toxicity, unwanted behavioural changes or any other devastating effect associated with psychiatric drugs. In another embodiment, a devastating effect includes an “off-target” effect.

In another embodiment, the present invention is aimed at assessing the effectiveness and the risks associated with the treatment of a bipolar disorder with a substance, a combination of substances, or a certain drug regimen. In another embodiment, the present invention is aimed at assessing the effectiveness and the risks associated with a “med cocktail”. In another embodiment, the present invention is aimed at assessing the effectiveness of a given substance in treating, reducing, and/or alleviating a specific symptom associated with a bipolar disorder or a specific set of symptoms associated with a bipolar disorder or schizophrenia. In another embodiment, the terms: “assessing”, “evaluating”, “testing” and all synonyms thereof are used interchangeably, herein. In another embodiment, the present invention can efficiently define indications/labels related to a bipolar disorder for a given compound or compounds that are being assessed according to the methods described herein.

In another embodiment, the present invention is aimed at utilizing the mouse model of the invention for targeting a certain symptom or a group of symptoms associated with a bipolar disorder or schizophrenia. In another embodiment, the present invention is aimed at utilizing the mouse model of the invention for identifying a substance or a combination of substances targeting a unique symptom or symptoms associated with a bipolar disorder or schizophrenia.

In another embodiment, the present invention is aimed at utilizing the mouse model of the invention for reducing the duration of a manic-like phase. In another embodiment, the present invention is aimed at utilizing the mouse model of the invention for reducing the duration of a depressive-like phase. In another embodiment, the present invention is aimed at utilizing the mouse model of the invention for reducing the number manic like phases. In another embodiment, the present invention is aimed at utilizing the mouse model of the invention for reducing the number of depressive-like phases. In another embodiment, the term “like” refers to a mouse behaviour homologous to a human behaviour. In another embodiment, the phrase “like phase” comprises a behaviour or a combination of behaviours in a mouse that are homologous to a human behaviour or a combination of human behaviours that is part of an illness, disease, or condition. In another embodiment, the phrase “depressive-like phase” comprises a behaviour or a combination of behaviours in a mouse that are homologous to a human depressive behaviour or a combination of human depressive behaviours.

In another embodiment, the present invention is aimed at utilizing the mouse model of the invention for increasing the efficiency of an already existing mood stabilizing drug by combining the drug with other substances or by optimizing the treatment regimen. In another embodiment, the present invention is aimed at utilizing the mouse model of the invention for reducing the dose required to efficiently treat patients with bipolar disorder.

### The Substance to be Evaluated

In another embodiment, a substance is a small molecule. In another embodiment, a substance is a peptide or a polypeptide. In another embodiment, a substance is a molecule. In another embodiment, a substance is a compound. In another embodiment, a substance is involved in a particular metabolic or signaling pathway that is specific to bipolar disorders. In another embodiment, a substance is a molecule which inhibits the functioning of a pathway in the diseased-bipolar state. In another embodiment, a substance is an organic small molecule. In another embodiment, a substance is a biopolymer-based drug (also known as biologics). In another embodiment, a substance is a nucleic acid such as but not limited to RNA for designing RNAi. In another embodiment, a substance is a drug having a non-psychiatric medical label such as, but not limited to, an anti-seizure drug wherein its efficacy as a mood stabilizer is evaluated according to the methods described herein.

In another embodiment, the efficacy of a substance to be used as a mood stabilizer is being evaluated according to the methods described herein. In another embodiment, the efficacy of a substance to be used as an antidepressant is being evaluated according to the methods described herein. In another embodiment, the efficacy of a substance to be used as an antipsychotic is being evaluated according to the methods described herein. In another embodiment, the efficacy of a substance to be used as an antianxiolytic is being evaluated according to the methods described herein. In another embodiment, the efficacy of a substance to be used as a sedative is being evaluated according to the methods described herein. In another embodiment, the efficacy of a substance to be used as an antimanic medication is being evaluated according to the methods described herein.

In one embodiment, the substance is a neuroleptic, which in one embodiment, is an antipsychotic medication.

In one embodiment, the substance is a typical antipsychotic, which in one embodiment, is Butyrophenone, which in one embodiment, is Haloperidol or Droperidol. In another embodiment, the substance is a Phenothiazine, which in one embodiment, is Chlorpromazine (Thorazine, Largactil), Fluphenazine (Prolixin), Perphenazine (Trilafon), Prochlorperazine (Compazine), Thioridazine (Mellaril, Melleril), Trifluoperazine (Stelazine), Mesoridazine, Periciazine, Promazine, Triflupromazine (Vesprin), Levomepromazine (Nozinan), Promethazine (Phenergan), Pimozide (Orap), or Cyamemazine (Tercian). In another embodiment, the substance is a Thioxanthene, which in one embodiment, is Chlorprothixene (Cloxan, Taractan, Truxal), Clopenthixol (Sordinol), Flupenthixol (Depixol, Fluanxol), Thiothixene (Navane), or Zuclopenthixol (Cisordinol, Clopixol, Acuphase). In one embodiment, the substance is an atypical antipsychotic, which in one embodiment, is Clozapine (Clozaril), Olanzapine (Zyprexa), Risperidone (Risperdal), Risperidone (Risperdal), Ziprasidone (Geodon), Amisulpride (Solian), Asenapine (Saphris), Paliperidone (Invega), Iloperidone (Fanapt), Zotepine (Nipolept, Losizopilon, Lodopin, Setous), Sertindole (Serdolect, Serlect), or Lurasidone (Latuda). In another embodiment, the substance is a third generation antipsychotic, which in one embodiment, is Aripiprazole (Abilify). In one embodiment, the substance is a partial dopamine agonist, a metabotropic glutamate receptor 2 agonist, a Glycine transporter 1 inhibitor, or a combination thereof. In one embodiment, the substance is Cannabidiol or Tetrabenazine. In another embodiment, the substance is lithium. In another embodiment, the substances to be evaluated are a combination of the substances described hereinabove, or a combination of a substance described hereinabove with another substance for treating a mental disorder, as described herein.

### Treatment

In another embodiment, treating is alleviating or reversing at least one symptom associated with a bipolar disorder. In another embodiment, treating is reducing the frequency or the magnitude of at least one symptom associated with a bipolar disorder. In another embodiment, treating is alleviating or reversing at least one symptom associated with the manic or hypomanic phase of a bipolar disorder. In another embodiment, treating is relieving at least one symptom associated with a bipolar disorder.

In another embodiment, treatment of a bipolar disorder comprises alleviating or reversing a symptom or symptoms associated with a bipolar disorder or schizophrenia. In another embodiment, treatment of a bipolar disorder comprises alleviating or reversing dramatic and unpredictable mood swings associated with a bipolar disorder or schizophrenia. In another embodiment, treatment of a bipolar disorder comprises reducing the frequency of dramatic and unpredictable mood swings or schizophrenia. In another embodiment, treatment of a bipolar disorder comprises alleviating or reversing excessive happiness associated with a bipolar disorder or schizophrenia. In another embodiment, treatment of a bipolar disorder comprises reducing the frequency of excessive happiness. In another embodiment, treatment of a bipolar disorder comprises alleviating or reversing excessive excitement associated with a bipolar disorder. In another embodiment, treatment of a bipolar disorder comprises reducing the frequency of excitement. In another embodiment, treatment of a bipolar disorder comprises alleviating irritability associated with a bipolar disorder or schizophrenia. In another embodiment, treatment of a bipolar disorder comprises reducing the frequency of irritability. In another embodiment, treatment of a bipolar disorder comprises alleviating restlessness associated with a bipolar disorder or schizophrenia. In another embodiment, treatment of a bipolar disorder comprises reducing the frequency of restlessness. In another embodiment, treatment of a bipolar disorder comprises alleviating or reversing symptoms such as increased energy, less need for sleep, racing thoughts, high sex drive, a tendency to make grand and unattainable plans, or any combination thereof. In another embodiment, treatment of a bipolar disorder comprises reducing the frequency of symptoms such as increased energy, less need for sleep, racing thoughts, high sex drive, a tendency to make grand and unattainable plans, or any combination thereof.

In another embodiment, treatment of a bipolar disorder comprises alleviating or reversing symptoms such as sadness, anxiety, irritability, loss of energy, uncontrollable crying, change in appetite causing weight loss or gain, increased need for sleep, difficulty making decisions, and thoughts of death or suicide, or any combination thereof. In another embodiment, treatment of a bipolar disorder comprises reducing the frequency of symptoms such as sadness, anxiety, irritability, loss of energy, uncontrollable crying, change in appetite causing weight loss or gain, increased need for sleep, difficulty making decisions, and thoughts of death or suicide, or any combination thereof. In another embodiment, treatment of a bipolar disorder comprises alleviating or reversing symptoms associated with a particular bipolar disorder.

### Bipolar Disorder

In another embodiment, the bipolar disorder is bipolar I. In another embodiment, the bipolar disorder is bipolar II. In another embodiment, the bipolar disorder is cyclothymic disorder. In another embodiment, the bipolar disorder is mixed bipolar. In another embodiment, the bipolar disorder is rapid-cycling bipolar disorder.

In another embodiment, a bipolar disorder includes symptoms such as euphoria, extreme optimism, inflated self-esteem, poor judgment, rapid speech, racing thoughts, aggressive behaviour, agitation or irritation, increased physical activity, risky behaviour, spending sprees or unwise financial choices, increased drive to perform or achieve goals, increased sex drive, decreased need for sleep, inability to concentrate, careless or dangerous use of drugs or alcohol, frequent absences from work or school, delusions or a break from reality (psychosis), poor performance at work or school, delusions, hallucinations, or any combination thereof.

In another embodiment, a bipolar disorder includes symptoms such as sadness, hopelessness, suicidal thoughts or behaviour, anxiety, guilt, sleep problems, low appetite or increased appetite, fatigue, loss of interest in daily activities, problems concentrating, irritability, chronic pain without a known cause, frequent absences from work or school, poor performance at work or school, seasonal changes in mood. In another embodiment, a bipolar disorder includes symptoms such as hedonic behaviour. In another embodiment, a bipolar disorder includes symptoms such as anhedonic behaviour.

In another embodiment, symptoms of a bipolar disorder vary according to the condition of the subject afflicted with the disease as described below. In another embodiment, a bipolar disorder includes at least one manic episode. In another embodiment, a bipolar disorder includes a period of abnormally elevated mood, accompanied by abnormal behaviour that disrupts the subject's life. In another embodiment, a bipolar disorder includes moods cycling between high and low over time. In another embodiment, a bipolar disorder includes frequent episodes of depression. In another embodiment, a bipolar disorder includes both mania and depression simultaneously or in rapid sequence. In another embodiment, a symptom associated with a bipolar disorder includes self-injury, often referred to as cutting, self-mutilation, or self-harm. In another embodiment, a bipolar disorder includes symptoms such as extreme anger, anxiety, and frustration. In another embodiment, a symptom or symptoms of a bipolar disorder are repetitive, not a one-time act. In another embodiment, a symptom or symptoms of a bipolar disorder are repetitive over a period of 6 months. In another embodiment, a symptom associated with a bipolar disorder can vary according to age and include in children and adolescents: explosive temper, rapid mood shifts, reckless behaviour and aggression.

### Administration

In another embodiment, administering the substance or a combination of substances to a mouse mutant ectopically expressing Otx2 in the hindbrain includes suitable routes of administration, for example, oral, rectal, transmucosal, transnasal, intestinal or parenteral delivery, including intramuscular, subcutaneous and intramedullary injections as well as intrathecal, direct intraventricular, intravenous, inrtaperitoneal, intranasal, or intraocular injections. In another embodiment, the preparation is administered in a local rather than systemic manner, for example, via injection of the preparation directly into a specific region of the mouse body.

Oral administration, in one embodiment, comprises a unit dosage form comprising tablets, capsules, lozenges, chewable tablets, suspensions, emulsions and the like. Such unit dosage forms comprise a safe and effective amount of the assayed compound. The pharmaceutically-acceptable carriers suitable for the preparation of unit dosage forms for peroral administration are well-known in the art. In some embodiments, tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. In one embodiment, glidants such as silicon dioxide can be used to improve flow characteristics of the powder-mixture. In one embodiment, coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. In some embodiments, the selection of carrier components depends on secondary considerations like taste, cost, and shelf stability, which are not critical for the purposes of this invention, and can be readily made by a person skilled in the art.

In one embodiment, the oral dosage form comprises predefined release profile. In one embodiment, the oral dosage form of the present invention comprises an extended release tablets, capsules, lozenges or chewable tablets. In one embodiment, the oral dosage form of the present invention comprises a slow release tablets, capsules, lozenges or chewable tablets. In one embodiment, the oral dosage form of the present invention comprises an immediate release tablets, capsules, lozenges or chewable tablets. In one embodiment, the oral dosage form is formulated according to the desired release profile of the pharmaceutical active ingredient as known to one skilled in the art.

Peroral compositions, in some embodiments, comprise liquid solutions, emulsions, suspensions, and the like. In some embodiments, pharmaceutically-acceptable carriers suitable for preparation of such compositions are well known in the art. In some embodiments, liquid oral compositions comprise from about 0.001% to about 0.933% of the assayed compound or compounds, or in another embodiment, from about 0.01% to about 1%.

In some embodiments, compositions for administration in the methods of this invention comprise solutions or emulsions, which in some embodiments are aqueous solutions or emulsions comprising a safe and effective amount of the compounds of the present invention.

In another embodiment, the pharmaceutical compositions are administered by intravenous, intra-arterial, or intramuscular injection of a liquid preparation. In some embodiments, liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intra-arterially, and are thus formulated in a form suitable for intra-arterial administration. In another embodiment, the pharmaceutical compositions are administered intramuscularly, and are thus formulated in a form suitable for intramuscular administration.

In one embodiment, pharmaceutical compositions for use in accordance with the present invention is formulated in conventional manner using one or more physiologically acceptable carriers comprising excipients and auxiliaries, which facilitate processing of the assayed compound (substance) into preparations which, can be used pharmaceutically. In one embodiment, formulation is dependent upon the route of administration chosen.

In one embodiment, injectables of the invention are formulated in aqueous solutions. In one embodiment, injectables of the invention are formulated in physiologically compatible buffers such as Hank's solution, Ringer's solution, or physiological salt buffer. In some embodiments, for transmucosal administration, penetrants appropriate to the barrier to be permeated are used in the formulation. Such penetrants are generally known in the art.

In one embodiment, the preparations described herein are formulated for parenteral administration, e.g., by bolus injection or continuous infusion. In some embodiments, formulations for injection are presented in unit dosage form, e.g., in ampoules or in multidose containers with optionally, an added preservative. In some embodiments, compositions are suspensions, solutions or emulsions in oily or aqueous vehicles, and contain formulatory agents such as suspending, stabilizing and/or dispersing agents.

The compositions also comprise, in some embodiments, preservatives, such as benzalkonium chloride and thimerosal and the like; chelating agents, such as edetate sodium and others; buffers such as phosphate, citrate and acetate; tonicity agents such as sodium chloride, potassium chloride, glycerin, mannitol and others; antioxidants such as ascorbic acid, acetylcystine, sodium metabisulfote and others; aromatic agents; viscosity adjustors, such as polymers, including cellulose and derivatives thereof; and polyvinyl alcohol and acid and bases to adjust the pH of these aqueous compositions as needed.

In some embodiments, pharmaceutical compositions for parenteral administration include aqueous solutions of the active preparation in water-soluble form. Additionally, suspensions of the active ingredients, in some embodiments, are prepared as appropriate oily or water based injection suspensions. Suitable lipophilic solvents or vehicles include, in some embodiments, fatty oils such as sesame oil, or synthetic fatty acid esters such as ethyl oleate, triglycerides or liposomes. Aqueous injection suspensions contain, in some embodiments, substances, which increase the viscosity of the suspension, such as sodium carboxymethyl cellulose, sorbitol or dextran. In another embodiment, the suspension also contain suitable stabilizers or agents which increase the solubility of the active ingredients to allow for the preparation of highly concentrated solutions.

In another embodiment, the assayed compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid).

In another embodiment, the pharmaceutical composition is delivered in a controlled release system is formulated for intravenous infusion, implantable osmotic pump, transdermal patch, liposomes, or other modes of administration. In one embodiment, a pump is used (see Langer, supra; Sefton, CRC Crit. Ref Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

In some embodiments, the assayed compound is in powder form for constitution with a suitable vehicle, e.g., sterile, pyrogen-free water based solution, before use. In one embodiment, the preparation of the present invention is formulated in rectal compositions such as suppositories or retention enemas, using, e.g., conventional suppository bases such as cocoa butter or other glycerides.

In some embodiments, pharmaceutical compositions suitable for use in context of the present invention include compositions wherein the assayed compounds are contained in an amount effective to achieve the intended purpose. In some embodiments, a therapeutically effective amount means an amount of active ingredients effective to prevent, alleviate or ameliorate symptoms of a bipolar disease or reduce the frequency of these symptoms. In one embodiment, determination of a therapeutically effective amount can be assessed by utilizing the methods of the invention and is also well within the capability of those skilled in the art.

The compositions also include incorporation of the assayed compound into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance.

In another embodiment, the methods of the invention are suitable for assessing/evaluating the in vivo toxicity and therapeutic efficacy of the assayed substances. In one embodiment, the data obtained from these in vivo is used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

In one embodiment, depending on the severity and responsiveness of the condition to be treated, dosing can be of a single or a plurality of administrations, with a course of treatment lasting from several days to several weeks or until a cure is effected or diminution of the disease state is achieved.

### Delivering Otx2 to the Brain

In one embodiment, Otx2 is delivered to the brain. In another embodiment, Otx2 is delivered to the hindbrain. In another embodiment, methods of delivering a gene and ectopically expressing the gene in an area of the brain are known to one of skill in the art. In another embodiment, methods of delivering Otx2 into the hindbrain comprise attaching an Otx2 transcript to a liposome such as a polyethylene glycol liposome. In another embodiment, methods of delivering Otx2 into the hindbrain comprise a viral delivery system such as a herpes simplex virus delivery system.

In another embodiment, methods of delivering Otx2 into the hindbrain comprise a vehicle that can penetrate through the blood-brain barrier (BBB). In another embodiment, the vector for delivering Otx2 according to the methods of the invention is a lentiviral vector. In another embodiment, the vector for delivering Otx2 according to the methods of the invention is an Adeno-associated virus. In another embodiment, Otx2 is delivered within a nanoparticle targeted to the hindbrain. In another embodiment, Otx2 is delivered by postnatal non-ventricular microinjection and in vivo electroporation which allows targeted delivery of Otx2 to a brain region. In another embodiment, Otx2 is delivered by in-utero electroporation. In another embodiment, methods of delivering Otx2 into the hindbrain comprise any method known to one of skill in the art.

### Mouse Bearing an Otx2 Gain-of-Function Mutation (a Mutation Leading to the Ectopic Expression of Otx2 in the Hindbrain

In another embodiment, the mouse of the invention is a mouse bearing an Otx2 gain-of-function mutation. In another embodiment, a mouse bearing an Otx2 gain-of-function mutation is the mouse described in Broccoli V. et al., 1999 (Vania Broccoli, Edoardo Boncinelli & Wolfgang Wurst. The caudal limit of Otx2 expression positions the isthmic organizer. Nature 401, 164-168 (9 Sep. 1999)), which is hereby incorporated by reference in its entirety. In another embodiment, methods of obtaining a mouse bearing an Otx2 gain-of-function mutation are described in Broccoli V. et al., 1999.

In another embodiment, the mouse bearing an Otx2 gain-of-function mutation is characterized by ectopic expression of Otx2 and phenotypic alterations of midbrain and cerebellum.

In another embodiment, a mouse bearing an Otx2 gain-of-function mutation is a mouse comprising a knock-in Otx2 functional gene. In another embodiment, a knock-in Otx2 mouse comprises an Otx2IRESlacZ (Otx2lacZ) inserted into the En1 locus. In another embodiment an Otx2 minigene which is not murine is inserted into the En1 locus.

In another embodiment, a mouse bearing an Otx2 gain-of-function mutation comprises a knock-in of a murine Otx2 minigene into the En1 locus. In another embodiment, preparation of a mouse bearing an Otx2 gain-of-function comprises: (1) the use of the En1 knock-in vector is utilized (Hanks, M., Wurst, W., Anson-Cartwright, L., Auerbach, A. B.& Joyner, A. L. Rescue of the En1 mutant phenotype by replacement of En1 with En2. Science 269, 679±682 (1995).), (2) the PGK-neo cassette which is flanked by loxP sites, allowing the removal of the neomycin gene cassette is used, (3) a mouse Otx2 complementary DNA encompassing the entire coding sequences followed by an encephalomyocarditis-virus-derived internal ribosome entry site (IRES) sequence linked to a bacterial lacZ reporter gene is inserted into the Kpnl site of the En1 knock-in vector, (4) placing the Otx2 minigene downstream of the En1 promoter, (5) R1 ES cells are then cultured, electroporated and selected, (6) recombinant clones are identified by Southern blotting using Hind111 and Xbal restriction enzymes, (7) as a 5′ probe, a 1.2-kb Sad fragment and, as a 3′ probe, a 700-bp EcoRI-Hind111 fragment are used to identify a 7-kb or 4.5-kb fragment after a proper homologous recombination event, (8) two independent targeted ES cell clones were injected into C57BL/6 blastocysts, (9) germline chimaeras are bred with either C57BL/6 or 129/Sv mouse strains, (10) germline transmission is detected by Southern blot analysis after Hind111 digestion of genomic DNA and hybridization with a 3′ probe, (11) F1 offspring are then mated with the deleter transgenic mouse line expression cre-recombinase ubiquitously, (12) cre-mediated neo excision is monitored by three PCR reactions detecting the presence of lacZ and ere and confirming the loss of the neo sequence.

In another embodiment, the mouse of the invention comprises an elevated amount of Otx2 protein in the hindbrain. In another embodiment, an elevated amount of Otx2 protein is ectopic expression of Otx2 in the hindbrain. In another embodiment, an elevated amount of Otx2 protein is ectopic expression of Otx2 in the hindbrain but not in other anatomical areas of the brain. In another embodiment, the mouse of the invention comprises an elevated amount of Otx2 protein in the hindbrain. In another embodiment, the mouse of the invention comprises an elevated amount of WT Otx2 protein in the hindbrain. In another embodiment, a mouse of the invention bears a mutation leading to the ectopic expression of Otx2 in the hindbrain. In another embodiment, a mouse of the invention bears a mutation leading to the ectopic expression of WT Otx2 in the hindbrain. In another embodiment, a mouse bearing a mutation leading to the ectopic expression of Otx2 in the hindbrain is a mouse comprising a knock-in of the Otx2 functional gene. In another embodiment, a knock-in Otx2 mouse comprises an Otx2IRESlacZ (Otx2lacZ) inserted into the En1 locus. In another embodiment, a mutation leading to the ectopic expression of Otx2 into the hindbrain comprises the entire sequence of the Otx2 gene or functional parts of this sequence inserted into the En1 locus. In another embodiment, a mutation leading to the ectopic expression of WT Otx2 into the hindbrain comprises the entire sequence of the Otx2 gene or functional parts of this sequence inserted into the En2 locus. In another embodiment, a mutation leading to the ectopic expression of Otx2 into the hindbrain comprises the entire sequence of the Otx2 gene or parts of this sequence inserted into an allele of a gene that is expressed in the developing hindbrain.

In another embodiment, the phrase: “elevated amount of Otx2 protein” is synonymous with the phrases “ectopic amount of Otx2 protein” and “ectopic expression of Otx2”.

In another embodiment, a knock-in Otx2 mouse is made by the use En1 Otx2lacZ targeting vector containing Otx2lacZ cassette, selectable thymidine kinase (TK) and a neomycin gene flanked by loxP sites. After gene targeting, Otx2lacZ is inserted into the En1 locus downstream of the promoter, deleting the first 111 amino acids of En1 and generating a null mutation. After cre recombinase activation, the neomycin selector gene is removed leaving a single loxP site. After germline transmission the F1 generation is bred with a cre deleter strain to remove the neo cassette.

In another embodiment, a knock-in Otx2 mouse is materially different than a knock-out mouse or a mouse with a disruption in the Otx2 gene. In another embodiment, the discovery that a knock-in Otx2 mouse serves as a valid model for a bipolar disorder is completely unexpected and is of immense advantage to the fields of neurobiology/psychiatry. In another embodiment, the discovery that a knock-in Otx2 mouse serves as a valid model for a bipolar disorder or schizophrenia is completely unexpected as one of skill in the art, based on the state of the art, is expected to utilize a mouse having reduced amounts of functional Otx2 protein or a mouse with a disrupted Otx2 gene in the hindbrain as a predictive model for assessing behaviours and treatments for bipolar disorders. In another embodiment, the discovery that a knock-in Otx2 mouse serves as a valid model for a bipolar disorder is completely unexpected as one of skill in the art, based on the state of the art, is expected to link polymorphisms in the OTX2 gene to bipolar disorders. In another embodiment, the discovery that a knock-in Otx2 mouse, expressing elevated and/or ectopic levels of WT and functional Otx2 protein in the hindbrain, serves as a valid model for a bipolar disorder is completely unexpected as one of skill in the art, based, on the state of the art, will assume that only the disruption of Otx2 gene in a mouse might induce bipolar-like behaviours in the mouse which result from cortical malformations and causes serotonergic and dopaminergic cells in the midbrain to be expressed in aberrant locations. In another embodiment, the discovery that a knock-in Otx2 mouse serves as a valid model for a bipolar disorder is completely unexpected as one of skill in the art, based on the state of the art, is expected to utilize a mouse bearing a polymorphic Otx2 allele and not a mouse with elevated and/or ectopic amounts of functional Otx2 protein in the hindbrain as a predictive model for assessing behaviours and treatments for bipolar disorders. In another embodiment, the current invention and discoveries further emphasize the complexity and unpredictability of: (1) molecular and developmental neuroscience in general; and particularly (2) the precise molecular mechanisms that underlie or can induce a bipolar disease, a bipolar behaviour, or a combination of behaviours that define a given bipolar disease. In another embodiment, there is a long felt need for a valid bipolar mouse model that can serve as an in vivo platform for assessing the efficacy of a bipolar disease treatment.

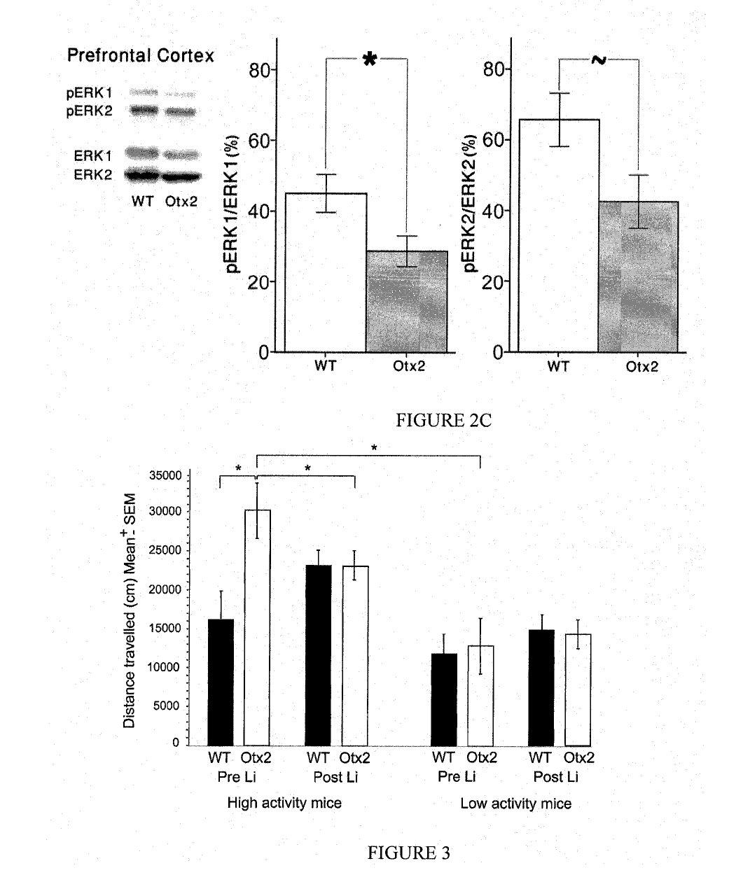

In another embodiment, Otx2 knock-in mouse of the invention express Otx2 under the endogenous En1 promoter, leading to an overexpression of Otx2 in the mid-hindbrain region. In another embodiment, Otx2 activates a signaling cascade involving Wnt1 and GSK-3 controlling the development of dopaminergic, serotonergic and noradrenergic neurons. In another embodiment, Otx2 knock-in mouse of the invention exhibits reduced serotonergic and noradrenergic neurotransmission. In another embodiment, Otx2 knock-in mouse of the invention have increased GSK-3 beta levels and reduced ERK levels in the hippocampus (FIG. 2).

### Evaluating Manic and Depressive-Like Behaviour

In one embodiment, evaluating manic and depressive-like behaviour is utilizing well accepted mouse models that mimic human manic and depressive behaviours. In another embodiment, well accepted mouse models that provide mouse manic and depressive behaviours are termed manic and depressive-like behaviour according to the invention. In another embodiment, evaluating manic and depressive-like behaviour is evaluating intra and/or inter-individual variation in the behaviour of mice of the invention. In another embodiment, evaluating manic and depressive-like behaviour is evaluating and measuring behaviour and “like-behaviours” such as but not limited to intra- and/or inter-individual variation in the behaviour of mice of the invention before and after administering the substance or substances of the invention.

In another embodiment, evaluating manic and depressive-like behaviour is evaluating and measuring behaviour and “like-behaviours” such as but not limited to intra- and/or inter-individual variation in the behaviour of the mice of the invention before and along treatment with the substance or substances of the invention. In another embodiment, evaluating manic and depressive-like behaviour is composed of before treatment (administration of a substance or substances as described herein) measure and/or evaluation and during treatment (hourly, daily, biweekly, or weekly) evaluations and measures of a behaviour and “like-behaviours” such as but not limited to intra- and/or inter-individual variation in the behaviour of mice of the invention. In another embodiment, evaluating manic and depressive-like behaviour is evaluating and measuring behaviour and “like-behaviours” such as but not limited to intra- and/or inter-individual variation in the behaviour of mutants in the short term (hours-one week). In another embodiment, evaluating manic and depressive-like behaviour is evaluating and measuring behaviour and “like-behaviours” such as but not limited to intra- and/or inter-individual variation in the behaviour of mutants in the long term (week-months). In another embodiment, evaluating manic and depressive-like behaviour is evaluating at least once before treatment and at least once after treatment commenced. In another embodiment, evaluating manic and depressive-like behaviour is evaluating before treatment until a stable individual baseline is reached and at least once after treatment commenced.

In another embodiment, the mouse homologous behaviour to human manic and depressive behaviour is termed manic and depressive-like behaviour. In another embodiment, locomotor activity was assessed by evaluating the distance mice traveled in the open field. In another embodiment, home cage activity was measured, as paradigm for activity levels in humans that are altered in patients afflicted with a bipolar disorder.

In another embodiment, mouse despair-like behaviour was assessed by the tail suspension test and/or the forced swim test. In another embodiment, time of immobility is used in both tests as a measure of a despair-like behaviour. In another embodiment, despair behaviour is altered in patients afflicted with a bipolar disorder.

In another embodiment, hedonic or anhedonic behaviour was assessed by the sugar or sucrose preference test. In another embodiment, the amount of sugar or sucrose consumed by the mice of the invention is used as a measure of hedonic/anhedonic like behaviour. In another embodiment, hedonic/anhedonic behaviour is altered in patients afflicted with a bipolar disorder or schizophrenia.

In another embodiment, anxiety is assessed by the elevated plus maze or time spent in the center of open field. In another embodiment, time spent in the open arm of the open field or in the center of the open field is used as a measure of anxiety and/or risk taking behaviour. In another embodiment, anxiety and/or risk taking behaviour are altered in patients afflicted with a bipolar disorder or schizophrenia.

In another embodiment, aggression is assessed by the resident intruder test. In another embodiment, an interaction such as latency: the time it takes to an intruder to be attacked by the resident is used as a measure of aggression like behaviour. In another embodiment, aggression is altered in patients afflicted with a bipolar disorder or schizophrenia.

In another embodiment, sleep is assessed by using EEG and EMG recordings. In another embodiment, time and ratio of sleeping phases are used as a measure for altered sleep. In another embodiment, altered sleep is a symptom of patients afflicted with a bipolar disorder or schizophrenia.

In another embodiment, rearing of mice is assessed by recording the behaviour of mice and evaluating the time and frequency of rearing manually or electronically. In another embodiment, stereotypic or repetitive behaviour of mice is assessed by recording the behaviour of mice and evaluating the time and frequency of stereotypic behaviour manually or electronically as is known in the art.

### Measuring Biological Markers as a Predictive Tool

In another embodiment, the severity of a bipolar disorder is further analyzed by measuring GSK-3, ERK and inositol signaling levels in the mouse. In another embodiment, measurement of GSK-3, ERK and inositol signaling levels in the mouse are preformed after evaluating manic and depressive-like behaviour in the mouse. In another embodiment, measurement of signal transduction pathways, known one of skill in the art, which is altered by mood stabilizing drugs are performed after evaluating manic and depressive-like behaviour in the mouse.

In another embodiment, any biological measurable marker, known to one of skill in the art, which correlates with a bipolar disorder can be analyzed after evaluating manic and depressive-like behaviour in the mouse In another embodiment, any biological measurable marker, known to one of skill in the art, which is altered in postmortem brains of patients with a bipolar disorder can be analyzed after evaluating manic and depressive-like behaviour in the mouse.

### The Subject

In another embodiment, a subject is a mouse or any other rodent. In another embodiment, a subject is a human subject afflicted with a bipolar disorder as described herein. In another embodiment, a subject is a child afflicted with a bipolar disorder as described herein. In another embodiment, a subject is an adolescent afflicted with a bipolar disorder as described herein. In another embodiment, a subject is an adult afflicted with a bipolar disorder as described herein.

In another embodiment, a subject is a human subject afflicted with a bipolar disorder as described herein combined with other disorders. In another embodiment, a subject is a human subject afflicted with schizophrenia.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

### Materials and Methods (Behavioural Studies)

### Locomotor Activity

WT and En1^{+/Otx2} littermates aged 4-6 month were tested in an open field box and locomotor activity was measured. Male and female mice were separately housed in groups (n=2-5) under standard laboratory conditions, with food and water ad libitum and a 12 hour light-dark cycle. Mice were placed in a transparent Perspex open field, measuring 40×40×20 cm. The box was cleaned with a dilute ethanol solution between animals and carefully dried. Locomotor activity was videotaped and analyzed off-line using the Noldus EthoVision® System.

### Hedonic or Anhedonic Behaviour Using the Sugar Preference Paradigm

Ten WT and 10 En1^{+/Otx2} male mice were taken as paired siblings from 10 litters and placed in a separate cage with ad lib access to food. First each cage was given two water bottles which were weighed before being placed in the cage and the following day, to confirm that the mice were sampling both bottles. After 12 hours of water deprivation, each mouse was given access to tap water in one bottle or an 8% sucrose solution in another bottle. The amount of fluid consumed in 12 hours was calculated by subtracting the weight of the bottle from its initial weight before it was placed in the cage. This procedure was repeated for 6 more days, alternating the side of the water and sucrose solutions each day. The data were analyzed by a 3 way ANCOVA for the effect of genotype, solution (within subject, water or sucrose) and days as a repeated measure. Because fluid consumption would be expected to be proportional to weight, the weight of the mice was used as a continuous variable

### Stereotypies and Rearing

Stereotypies and rearing were analyzed manually by an observer blind to the treatment by sampling for 60 s at the start of each 10 min interval and measuring the duration of stereotyped behaviour. Stereotypical behaviours were defined as sniffing while immobile, grooming, licking the bottom or wall of the box, head nodding or repetitive lateral H. (Tilleman et al./Neuroscience 163 (2009) 1012-1023 1013 head movements). The total duration of stereotyped behaviour and rearing was compared between the mutant and WT mice using a three-way ANOVA for the effect of treatment (three levels) and genotype (two levels) and time as a repeated measure.

### Center/Periphery Ratio

In order to define the center/periphery exploration ratio, we defined the center area in the Noldus Ethovision® system (20_{—}20 cm). Subsequently, the distances covered by the mice in the center and in the periphery were correlated with the total distance covered by the animals. Due to the fact that the number of mice in each experiment was too small to allow correlation analysis, the data from all saline-treated mice from the above three experiments were pooled.

### Elevated Plus Maze (EPM)

Thirteen En1/Otx2 (six females) and 19 WT (eight females) were tested on the EPM, which is a pharmacologically validated anxiety measure in rodents (File, 2001). The EPM was constructed of transparent Plexiglas and consisted of two opposing open arms and two opposing closed arms. The arms were 50 cm in length and 5 cm wide. The closed arms had walls that were 20 cm high, whereas the open arms had a ledge of 2 cm to prevent falling and the entire maze was elevated 50 cm from the floor. At the start of the trial, the mouse was placed in the center of the maze, facing an open arm and left to explore for 6 min. The trials were taped and duration spent in each of the arms was analyzed off line using the Noldus Ethovision® system.

### Tail Suspension Test

The tail suspension test (TST) was administered according to standard procedure between 11:00 A.M. and 1:00 P.M. Briefly, each mouse was suspended by the tail at a height of 40 cm for 6 min, during which immobility was measured by an experimenter blind to treatment using AnyMaze software (Stoelting). An animal was considered to be immobile when it did not show any movement and hanged passively.

### Forced Swim Test

Forced swimming test We used the modified FST, which is a modified version of the traditional rat FST (Porsolt et al. 1979) and was designed to increase sensitivity for detecting SSRIs (Detke et al. 1995, 1997). Briefly, mice were placed into white opaque plastic buckets (61 cm high, 48 cm diameter) filled 48 cm high with 23-25° C. tap water for 6 min each on two consecutive days. Swim sessions were videotaped from a tripodmounted camera positioned directly above swim buckets. Animals' behaviour was analyzed by a trained observer blind to treatment conditions using a time sampling technique in which the predominant behaviour (swimming, immobility, or climbing) is scored every 5 s for the last 4 min of the test (Cryan and Lucki 2000). Reductions in immobility and/or increases in swimming or climbing reflect antidepressant effects (Detke et al. 1997).

### Resident-Intruder Paradigm

The resident-intruder paradigm was used to study internale aggression. En1^{+/Otx2} mice serve as residents and wild-type mice serve as intruders. During the dark cycle an intruder mouse was introduced into the home cage of a resident mouse for 15 min. The session was videotaped and scored at a later date by an experienced rater. The following resident behaviours were measured: fighting (the incidence and duration of bouts), aggressive postures (sideways postures and tail rattling), and pursuit episodes. We also measured the time spent engaged in non-aggressive contact (exploratory sniffing) of the intruder.

### Sleep Analysis

Using EEG and EMG recordings a 24 hours spontaneous sleep-wake patterns are monitored.

### Novelty Suppressed Feeding Test

Twenty-four hours before the test, each mouse was weighed and then deprived of all food in their home cage. Prior to test, a food pellet was placed on a round filter paper (10 cm) and positioned in the center of the apparatus (40×40×40 cm). The subject mice were weighed again and the difference of body weight loss was assessed. The test began immediately after each subject was placed in the corner of the apparatus and latency to feed the pellet was measured by counting manually. The cutoff time of this test was set to 5 min. immediately, after attempting to feed or 5 min, each subject was transferred to its home cage and the amount of food eaten was measured by weighing pre- and post fed food pellet for 5 min to evaluate their appetite.

### Sensory Gating/Prepulse Inhibition

Measurement of acoustic startle response and prepulse inhibition Mice were placed in a plastic cylinder and fixed in an automated startle chamber (SR-Lab Systems, San Diego, Calif., USA). After a 5-min acclimation period with 70-dBbackground noise (white noise), an 75-, 80-, 85-, 90-, 100-, 110-, or 120-dB white noise stimulus (40 msec duration) was given 8 times to each mouse in the same pseudo-random order at 15 sec intervals. Analysis for startle amplitudes was based on the mean of the seven trials (ignoring the first trial) for each trial type. PPI responses were measured with 120 dB acoustic stimuli combined with four different prepulse intensities. Each mouse was placed in the startle chamber (SR-Lab) and initially acclimatized for 5 min with background noise alone (70 dB white noise). The mouse was then subjected to 48 startle trials, each trial consisting of one of six conditions: (i) a 40 msec 120 dB noise burst presented alone, (ii-v) a 40 msec 120 dB noise burst following prepulses by 100 msec (20 msec noise burst) that were 3-, 6-, 9-, or 12-dB above background noise (i.e., 73-, 76-, 79-, or 82-prepulse, respectively), or (vi) no stimulus (background noise alone), which was used to measure baseline movement in the chamber. These six trial types (i-vi) were each repeated 8 times in a pseudorandom order to give 48 trials. The inter-trial interval was 15 sec. Each trial type was presented once within a block of six trials and the order of 48 trial presentations was fixed for all mice. Analysis was based on the mean of the seven trials for each trial type. The percentage PPI of a startle response was calculated as 100−[(startle response on prepulse-pulse stimulus trials−no stimulus trials)/(pulse-alone trials−no stimulus trials)]×100.

### Five-Choice Serial Reaction Time Test.

The 5-CSRTT was conducted in operant conditioning test chambers (Med Associates) measuring 21.6×17.8×12.7 cm. One curved, stainless steel wall of the chamber contained five round apertures measuring 1.3 cm in diameter that were 1 cm deep and located 0.8 cm above the floor. An infrared photodetector was located at the entrance to each aperture, and a yellow stimulus light diode was centered at the back of each aperture. The opposite wall of the chamber was also constructed of stainless steel. A reinforcer magazine, equipped with an infrared photodetector and a light, was centered in this wall 2.5 cm above the floor. A motor-driven dipper arm could be raised to deliver 0.01 ml of 10% (w/v) sucrose solution to the magazine. Each box was illuminated by a house light and was enclosed in a sound-attenuating chamber equipped with a ventilation fan. All boxes were controlled by a personal computer running Med-PC-IV software (Med Associates).

### Example 1

### Behavioural Alterations (Bipolar-Like Behaviours) of Otx2 Gain of Function Mutants are Homologous to Bipolar Behaviours

Psychostimulant-induced hyperactivity is a well established model for mania. In the current experiments it was found that locomotor activity, stereotypic behaviour and rearing in saline-treated Otx2 mutants were similar to amphetamine- or methylphenidate-treated WT mice.

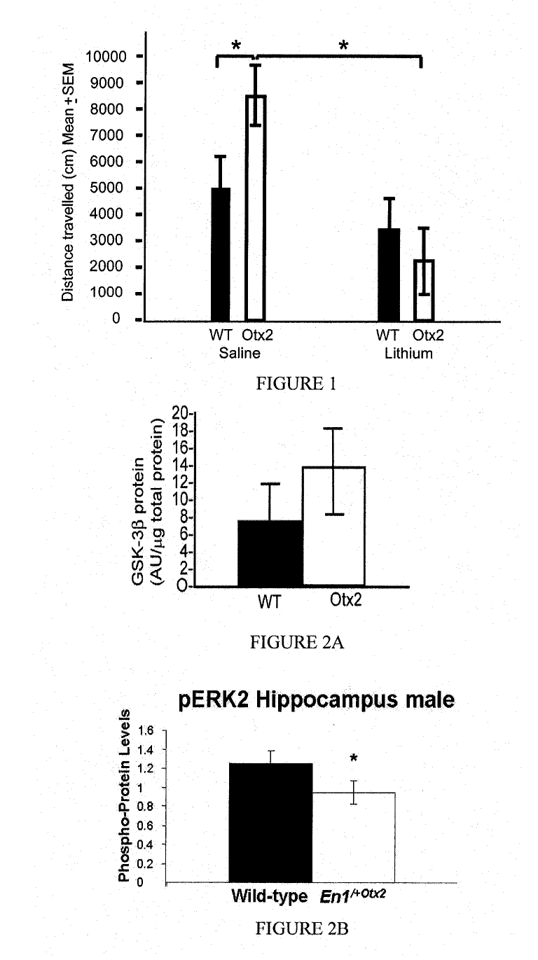

The results also showed an increase in GSK3-beta levels in mutants and emphasized the essential role of GSK-beta in monoaminergic neurotransmitter mediated behaviour (FIG. 2). This experiment utilized lithium, which blocks GSK-3 beta activity. Upon lithium treatment, it was found that hyperactivity of Otx2 mutants was reduced (FIG. 1).

Interestingly, it was found that the degree of activity among mutants is much more variable than among WT, suggesting a high inter-individual behaviour diversity of mutants. Locomotor activity of 26 WT and 26 mutants was assessed for 30 min. Six WT and 6 mutants, displaying the highest and lowest locomotor activity, were designated, respectively high and low activity groups. A significant difference between the average of the high and low activity groups in mutants, in contrast to WT was observed (p<0.05) (FIG. 3).

In order to assess whether lithium can reverse hyperactivity of the high activity group of Otx2 mice, WT and Otx2 mutants were exposed to chronic lithium treatment. It was found that lithium reduced the hyperactivity of Otx2 mice to levels comparable to WT (FIG. 3). In addition, lithium treatment also reduced the difference in locomotor activity of mutants with high and low activity to levels comparable to WT (FIG. 3). This suggests that the inter-individual variability in the behaviour of Otx2 mice can be reversed by exposure to lithium.

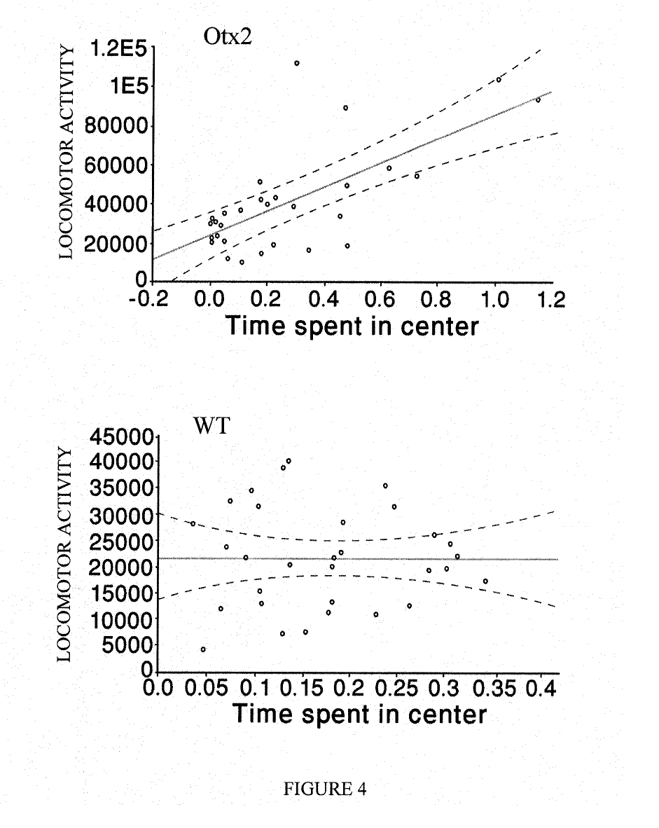

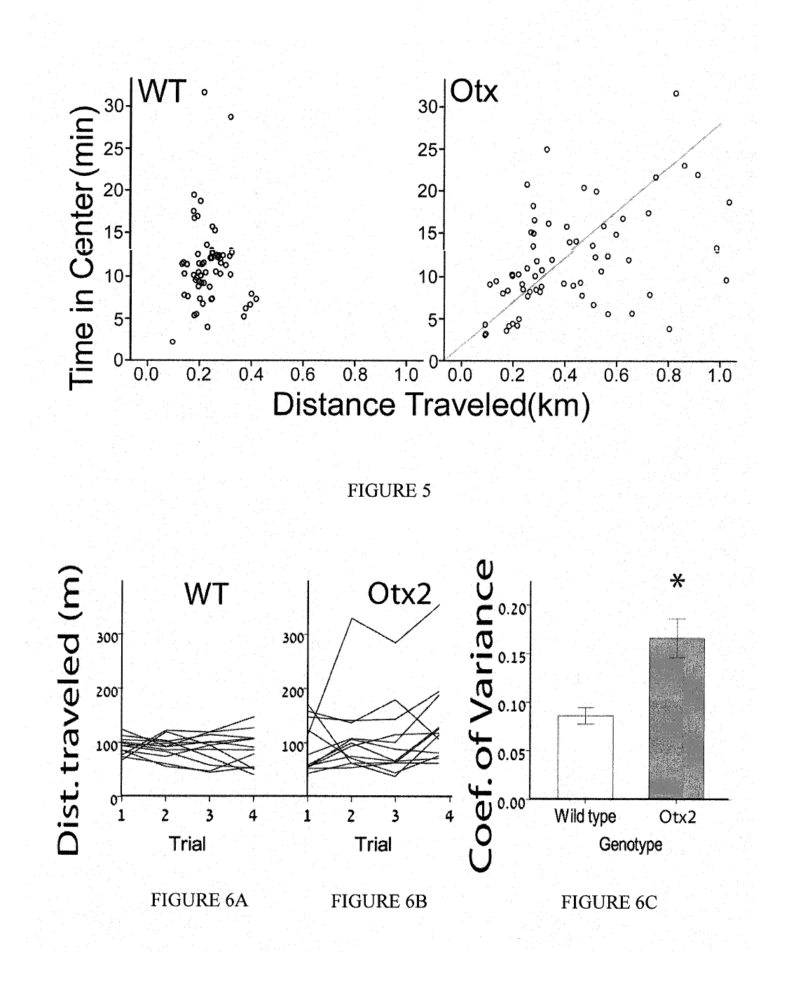

Next, a correlation between the activity of the mutant mice and the time spent in the center of the open field, a measure of anxiety-risk taking behaviour was tested. It was found that hyperactive Otx2 mice spent more time in the center of the open field, compared to WT mice, suggestive of mania related risk-taking behaviour (FIG. 4). The time Otx2 mutants spend in the center of the open field correlates with their locomotor activity levels (p<0.001), in contrast to WT (FIG. 5). We conclude that hyperactivity is associated in mutants with increased risk taking behaviour.

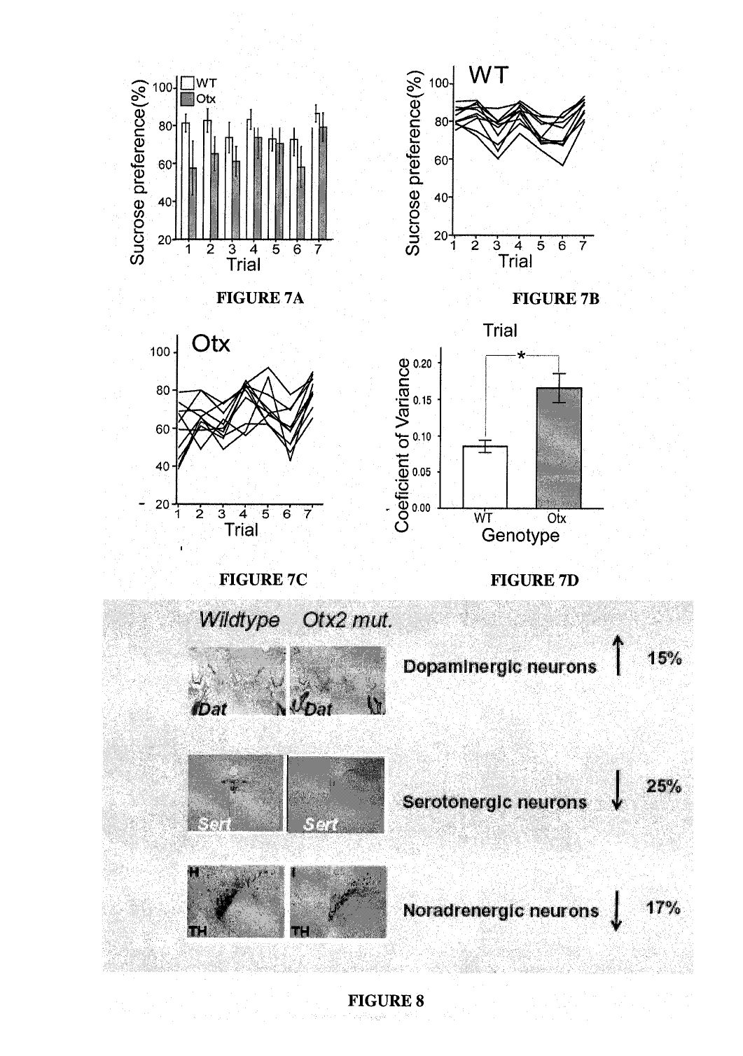

In order to assess whether Otx2 mice also show intra-individual behavioural variability over time, animals locomotor activity (FIG. 6) and sugar consumption (FIG. 7) were measured, the latter used as one measure for hedonic/anhedonic behaviour. Locomotor activity was assessed in 4 consecutive trials over a period of 12 days. In order to quantify intra-individual variability, the coefficient of variance was calculated by dividing the standard deviation of the mean distance traveled by individual animals by the mean distance. Comparing the coefficient of variance of mutants to WT mice, revealed an increased variability over time in locomotor activity of Otx2 mutant mice (FIG. 6). Comparing sugar intake of Otx2 compared to WT animals, using the same statistical method, revealed a significant increase in the coefficient of variance in sugar intake in mutants (FIG. 7), suggesting a higher variability over time also for this behavioural parameter.

Dopaminergic neurons develop during embryogenesis in the midbrain, close to the mid-hindbrain border. The activity of the mid-hindbrain organizer is mediated via the secreted molecules FGF8 and WNT1, while the transcription factor, OTX2, which is expressed in the midbrain, plays a crucial role in its positioning. The current Otx2 knock-in mutant mice ectopically express Otx2 in the hindbrain during embryogenesis, which leads to an aberrantly positioned mid-hindbrain organizer. The resulting mutants demonstrate abnormal brain development manifesting as decreased volume of the cerebellar vermis and an increase in the dopaminergic cell population. In adulthood these mutants exhibit pronounced overactivity of the dopaminergic system and are hyperactive as a result of factors that also underlie certain behaviours associated with Schizophrenia.

In addition to the long felt need for an animal model that mimics human behaviours associated with psychiatric disorders and/or disease, the present invention provides that the actual usefulness of an Otx2 mutant as described herein as a model to test effectiveness of mood stabilizing drugs is unexpected in view of the state of the art.

Three main categories of animal models exist: (1) Construct validity, assesses how much a model is consistent with the etiology and pathophysiology of a disease; (2) Face validity, assesses how similar the model is to the symptoms of a condition; and (3) Predictive validity, assesses how well a model responds to appropriate, therapeutics. The present data unexpectedly provides a model that shows multiple types of validity. This is unexpected and of great significance as many animal models show face and construct validity but no predictive validity. Therefore, the present data unexpectedly provides that the Otx2 mutant mouse model serves as a desired predictive validity model for assessing the efficacy of different treatments of psychiatric disorders and/or diseases. It is important to notice that a model having only face and construct validity is not sufficient for assessing the efficacy of treatment as provided herein. Previous “Gene-behaviour interactions” models such as described in Tilleman H. et al., 2009 (Tilleman H, Kofman O, Nashelsky L, Livneh U, Roz N, Sillaber I, Biegon A, Rehavi M, Brodski C. Critical role of the embryonic mid-hindbrain organizer in the behavioural response to amphetamine and methylphenidate. Neuroscience. 2009 Nov. 10; 163(4):1012-23) are limited to face validity and construct validity and do not provide predictive validity of the model which is utmost essential for assessing the efficacy of a given treatment in an animal model for a disorder/disease.

Having a bipolar disorder because of an Otx2 mutation in humans or having a manic and depressive-like behaviour in a mouse because of a Otx2 mutation such as described herein does not mean that the mouse will respond to a mood stabilizing drugs such as lithium. The predictive validity proved herein is based on the predictive response of the mice to a mood stabilizing drugs such as lithium.

### Example 2

### Otx2 Mouse Mutants are a Promising Model for Bipolar Disorder

Dopamine transporter (Dat), Serotonin transporter (Sert), and tyrosine hydroxylase (TH) were assessed using immunohistochemistry to identify cells expressing dopamine, serotonin, and norepinephrine, respectively in Otx2 mutant mice compared to wild type mice. There was a 15% increase in dopaminergic neurons in Otx2 mutant mice compared to wild type, and a 25% and 17% decrease in serotonergic and noradrenergic neurons, respectively, in Otx2 mutant mice (FIG. 8). Therefore, adult Otx2 mutants show alterations in monoaminergic neurons.

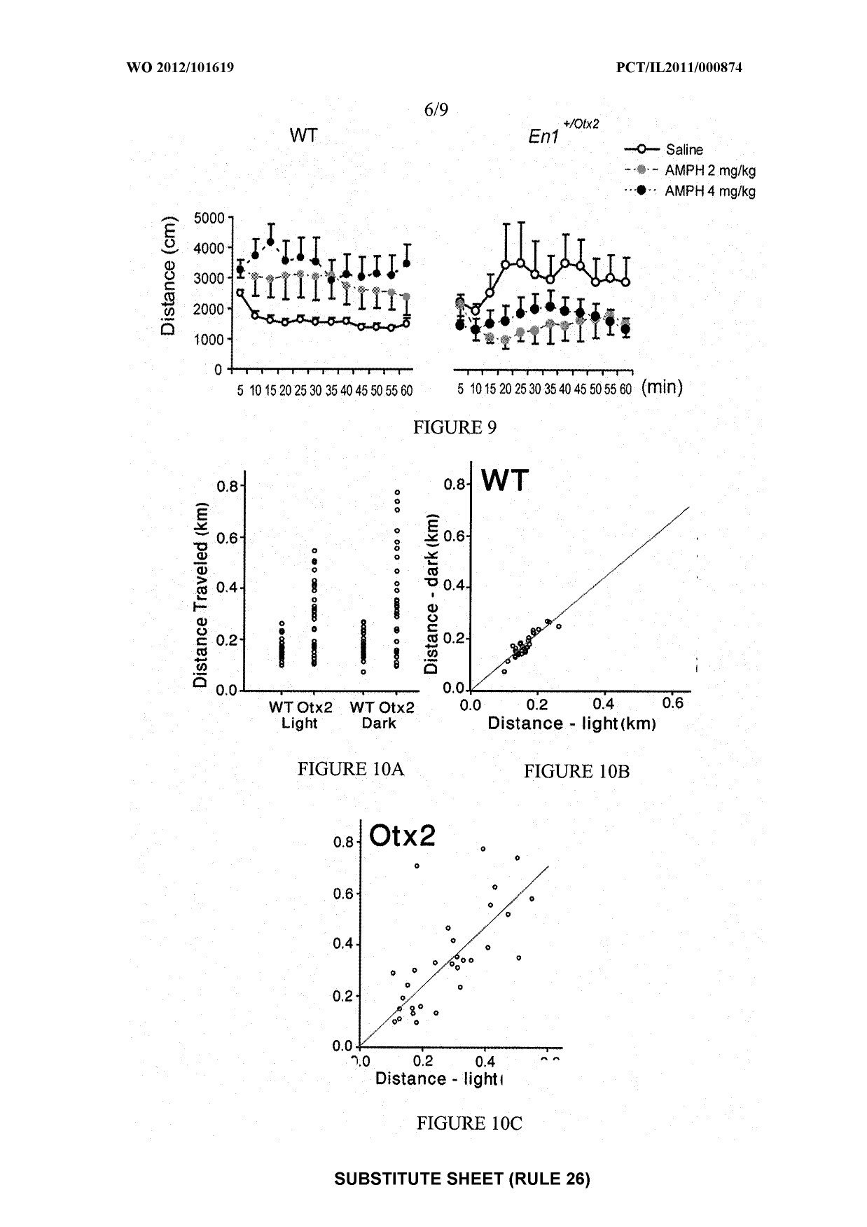

Otx2 mutants and wild type mice were evaluated for manic-like behaviour in the open field test for 60 min with or without a 2 mg/kg or 4 mg/kg administration of amphetamine. Saline-treated Otx2 mutants demonstrated a manic-like behaviour in the open field, similar to that of amphetamine-treated WT mice (FIG. 9).

Next, Otx2 and wild type (WT) animals were evaluated for hyperactivity in the open field test (FIG. 10). 30 Otx2 and 30 wild type (WT) animals were recorded for 45 min in the open field during both light and dark phase. Otx2 mutants were hyperactive (F=40.889, p<0.001) in both phases and showed more inter-individual variation (A). Distances traveled in the light phase correlated with the distances traveled in the dark phase for both WT (B) and Otx2 animals (C).

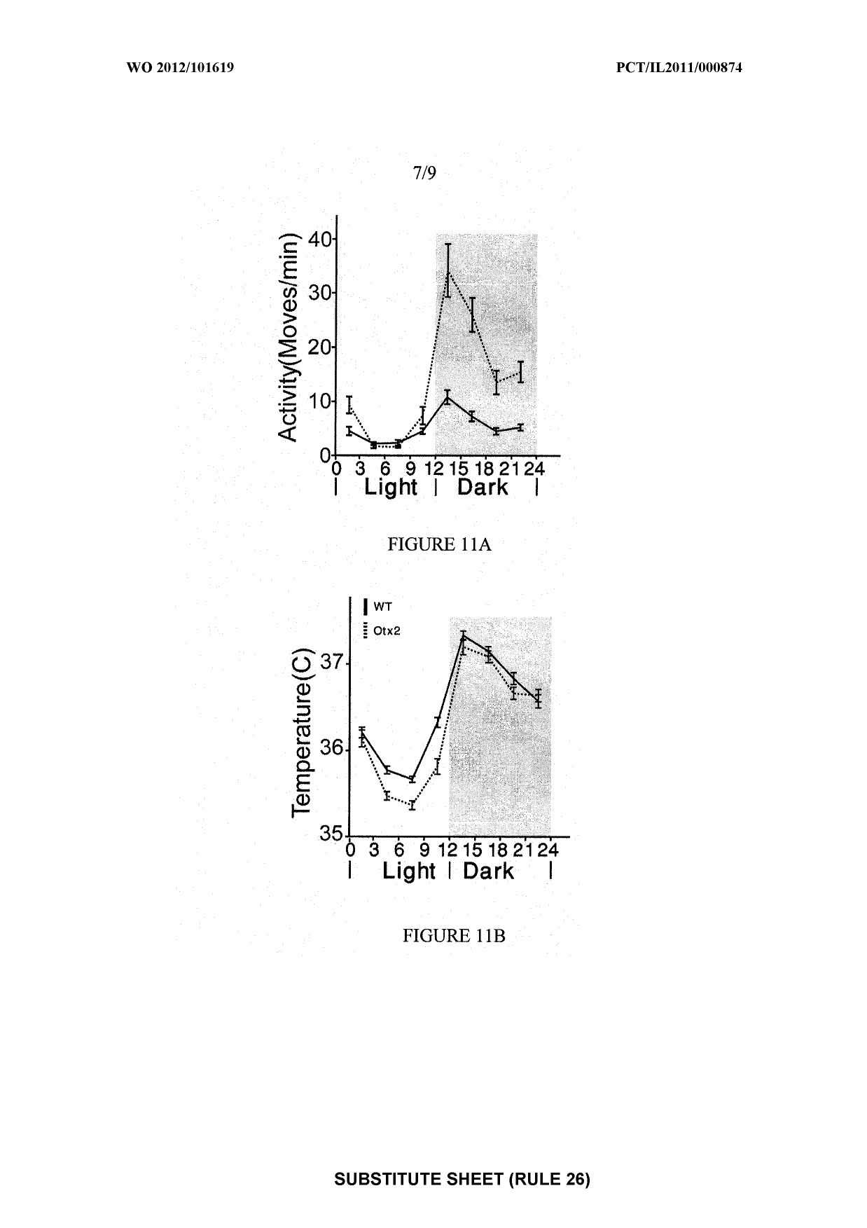

When Otx2 and wild type (WT) animals were evaluated for hyperactivity in their home cage using chronically implanted activity and body temperature monitor probes (FIG. 11), Otx mutants demonstrated hyperactivity in the dark phase compared to WT littermates. In the light phase, Otx2 mutants had decreased body temperature compared to controls.

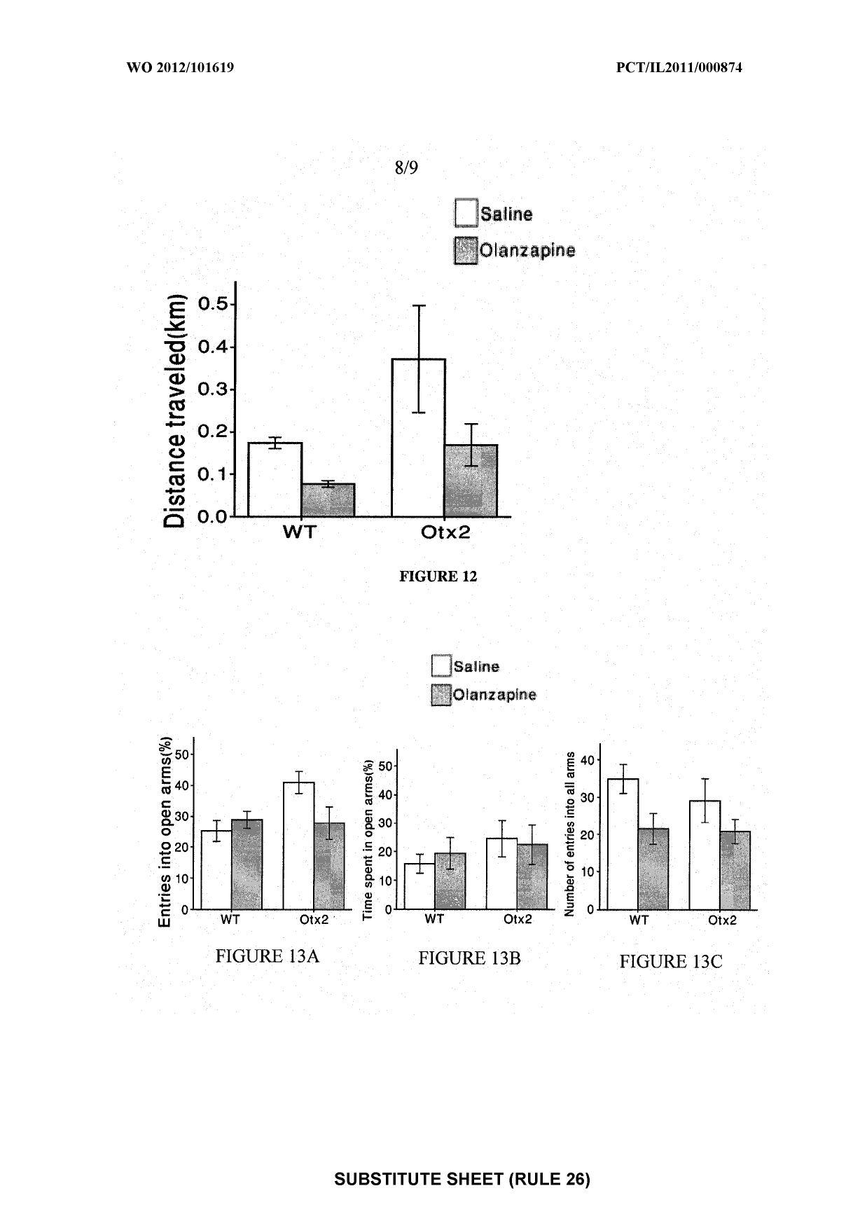

The hyperactivity demonstrated by Otx2 mutants was abrogated after s.c. injection with the antipsychotic drug Olanzapine (1 mg/kg; FIG. 12). Olanzapine also reduced the activity of WT mice (FIG. 12).

In the elevated plus maze, Otx2 mutants showed more entries into the open arm of the maze, which is a risk-taking behaviour. This behaviour is reversed in Otx2 mutants by olanzapine, indicating normalization of anxiety-like behaviour (FIG. 13).

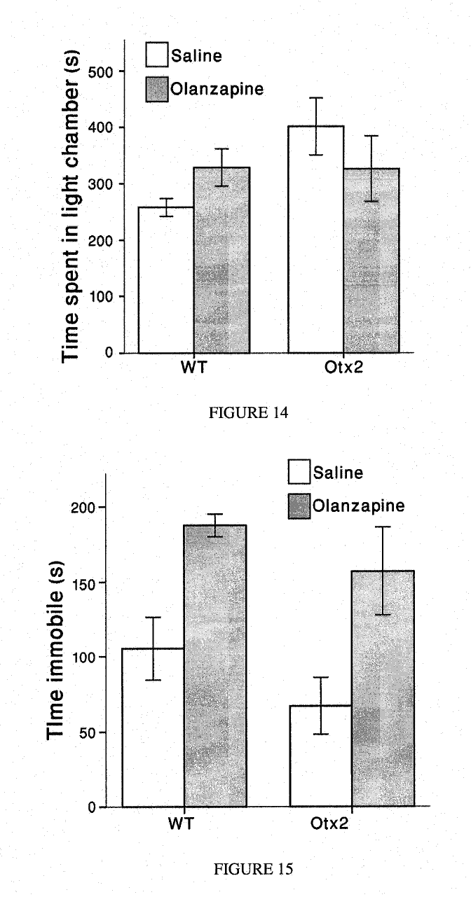

Using the dark-light box behavioural assay, Otx2 mutants again showed increased risk-taking behaviour by spending more time in the illuminated compartment (FIG. 14). Olanzapine reduced the time Otx2 mutants spent in the light compartment, and increased the time WT animals were present in the light compartment (FIG. 14).

Finally, in the forced swim test (FST), administration of Olanzapine to both Otx2 and WT mice increased immobility time for both groups (FIG. 15).

A significant bottle neck for the development of new drugs for bipolar disorder is the lack of appropriate animal models that may be used to evaluate the efficiency of potential new drugs. We provide additional evidence hereinabove supporting Otx2 mouse mutants as a model to evaluate drugs for bipolar disorder.

1. We found that Otx2 mutants are hyperactive during the first phase of the light cycle, suggesting that they sleep less. This is in accordance with patients with bipolar disorder showing reduced sleep in the manic phase.

2. We found that Otx2 mutants show more entries into the open arm of the elevated plus maze and into the light compartment of the dark-light box. These findings, indicating that Otx2 mutants show an increase in risk taking behaviour, which parallels the behaviour of patients in the manic phase.

3. Importantly, we found that olanzapine, which is used for the acute treatment of mania, can reverse the risk taking behaviour showed by the mutants.

Otx2 mouse mutants are therefore a promising model for Bipolar Disorder.

What is claimed is:

1. A method for evaluating the efficacy of a substance or a combination of substances for the treatment of a bipolar disorder in a subject, comprising the steps of:
a. administering said substance or said combination of substances to a mouse comprising an elevated amount of Otx2 protein in the hindbrain; and
b. evaluating manic and depressive-like behaviour in said mouse.

2. The method of claim 1, wherein said mouse comprising an elevated amount of Otx2 protein in the hindbrain bears an Otx2 gain-of-function mutation.

3. The method of claim 1, further comprising the step of measuring GSK-3, ERK and inositol signaling levels in said mouse after step (b).

4. The method of claim 1, wherein said evaluating manic and depressive-like behaviour comprises evaluating locomotor activity, hedonic behaviour, anhedonic behaviour, despair-like behaviour, sleep behaviour, aggression, anxiety, or a combination thereof.

5. The method of claim 4, wherein said locomotor activity is evaluated in an open field, in a home cage or in a running wheel.

6. The method of claim 4, wherein said despair like behaviour is evaluated by the tail suspension test, the forced swim test, or a combination thereof.

7. The method of claim 4, wherein said hedonic or anhedonic behaviour is evaluated by the sugar or sucrose preference test.

8. The method of claim 4, wherein said anxiety is evaluated by the elevated plus maze test, time spend in the center of open field test, or a combination thereof.

9. The method of claim 4, wherein said aggression is evaluated by the resident intruder test.

10. The method of claim 4, wherein said sleep behaviour is evaluated using EEG and EMG recordings during sleep phases.

11. The method of claim 4, wherein said depressive-like behaviour is evaluated by novelty suppressed feeding.
